(19) United States
(12) Patent Application Publication
(10) Pub. No.: US 2017/0151240 A1
(43) Pub. Date: Jun. 1, 2017

(54) F10 Inhibits Growth of PC3 Xenografts and Enhances the Effects of Radiation Therapy

(71) Applicant: WAKE FOREST UNIVERSITY, Winston-Salem, NC (US)

(72) Inventor: William H Gmeiner, Yadkinville, NC (US)

(21) Appl. No.: 15/316,160

(22) PCT Filed: Jun. 3, 2015
(86) PCT No.: PCT/US15/34024

§ 371 Date: Dec. 3, 2016

Related U.S. Application Data
(60) Provisional application No. 62/007,176, filed on Jun. 3, 2014.

(51) Int. Cl. A61K 31/505; A61K 45/06

(52) U.S. Cl. CPC A61K 31/505; A61K 47/48084; A61K 45/06

(57) ABSTRACT

Chemotherapy remains of limited use for the treatment of prostate cancer with only one drug, docetaxel, demonstrating a modest survival advantage for treatment of late-stage disease. Data from the NCI 60 cell line screen indicated that the castration-resistant prostate cancer cell lines PC3 and DU145 were more sensitive than average to the novel polymeric fluoropyrimidine (FP), F10, despite displaying less than average sensitivity to the widely-used FP, 5FU. In an embodiment of the present invention, F10 treatment of PC3 xenografts results in a significant survival advantage (treatment to control ratio (T/C) days=18; p<0.001; n=16) relative to control mice treated with saline. F10 (40 mg/kg/dose) was administered via jugular vein catheterization 3-times per week for five weeks. This aggressive dosing regimen was completed with no drug-induced weight loss and with no evidence of toxicity. F10 was also shown to sensitize PC3 cells to radiation and F10 was also shown to be a potent radiosensitizer of PC3 xenografts in vivo with F10 in combination with radiation resulting in significantly greater regression of PC3 xenografts than radiation alone. The results indicate that F10 in this pre-clinical setting is an effective chemotherapeutic agent and possesses significant radiosensitizing properties.

The present invention claims priority under 35 USC 365 and 371 to PCT Application PCT/US15/34024 filed Jun. 3, 2015 and under 35 USC 119(e) to U.S. Provisional Application No. 62/007,176 filed Jun. 3, 2014, the entire contents of all of which are incorporated by reference in their entireties.

The present invention was supported by DOD PCRP 093606, DOD PCRP 110135, NIH CA102532, NIH P30 CA12197 and the NCI RAID Program. Accordingly, the federal government has rights in the invention.

### FIELD OF THE INVENTION

The present invention relates to compounds and compositions for treating cancer and methods associated therewith. For example, the compounds, compositions and methods of the present invention can be used in the treatment of prostate cancer such as castration-resistant prostate cancer.

### BACKGROUND OF THE INVENTION

Prostate cancer is the most frequently diagnosed cancer in men accounting for nearly one-third of all new malignancies in American men and nearly 30,000 deaths. Chemotherapy has limited utility for the treatment of prostate cancer although docetaxel given in combination with prednisone or estramustine provides a survival benefit for treatment of castration-resistant prostate cancer (CRPC). Docetaxel as an adjuvant to radical prostatectomy also provides a survival advantage for men at high-risk for recurrent disease although treatment caused serious toxicities. There is an urgent need for new and more effective chemotherapeutic options with fewer side effects for prostate cancer patients.

### SUMMARY OF THE INVENTION

F10 is a novel polymeric fluoropyrimidine (FP) that is under pre-clinical development for treatment of acute myeloid leukemia (AML), glioblastoma (GBM) and other malignancies (FIG. 1A). The cytotoxic mechanism of F10 involves dual targeting of thymidylate synthase (TS) and DNA topoisomerase 1 (Top1) causing replication-mediated DNA double-strand breaks (DSBs).

F10 mechanistically resembles the camptothecin (CPT) class of anticancer drugs and is primarily directed towards the DNA locus of FP activity. Results from the NCI-60 cell line screen demonstrated mechanistic similarities to other Top1 poisons but unexpected mechanistic dissimilarities to 5-fluorouracil (5-FU) which is cytotoxic by an RNA-mediated process. The NCI-60 data also showed that the CRPC cell lines PC3 (a human prostate cancer cell line) and DU145 cell lines were nearly 1,000-fold more sensitive to F10 than 5-FU suggesting that F10 might be effective for treating CRPC even though 5-FU and capecitabine (a 5-FU pro-drug) are not. F10 is very well-tolerated in vivo, in part because of high specificity for proliferating cells, and may be efficacious without reducing patient quality of life.

In addition to their use in chemotherapy, FPs and Top1 poisons have significant radiosensitizing properties. As radiation therapy is used for treating localized prostate cancer and preventing disease recurrence, information on the radiosensitization properties of F10 is important for translational efforts. The present invention demonstrates that F10 inhibits the growth of PC3 xenografts as a single agent and that the combination of F10 and radiation synergistically inhibits growth of PC3 cells in tissue culture and in vivo. PC3 cells are derived from bone metastases and have been used as a cellular model of CRPC. The present invention demonstrates that F10 is extremely well-tolerated in vivo with much more extensive dosing possible with F10 than with widely-used FPs, such as 5FU. The results obtained indicate that F10 should be evaluated for in vivo efficacy and radiosensitization for the clinical treatment of many types of cancers, for example, prostate cancer.

### BRIEF DESCRIPTION OF THE DRAWINGS

FIG. 1 shows that F10 strongly inhibits the clonogenicity of PC3 cells. (A) Structure of F10. (B) Clonogenic survival assay evaluating clonogenic survival of PC3 cells following 72 h treatment with either F10 or 5-FU at the indicated concentration (nM). No surviving colonies were observed following treatment with F10 at 1 μM (1,000 nM). In contrast, treatment with 5FU at 10 μM (10,000 nM) decreased the percent of colonies surviving relative to control by less than 50%. (C) TS activity in PC3 cells at the indicated times following treatment with F10 (10⁻⁸ M) or 5-FU (10⁻⁶ M) (*p<0.05 vs control based on Student's two sided t-test).

FIG. 2 shows results of a clonogenic assay evaluating the survival fraction of PC3 cells exposed to radiation or radiation in combination with F10. (A) Graph of surviving fraction as a percent of non-treated cells. (B) Same data as in (A) but normalized for F10-only effects to determine if F10 were radiosensitizing. (*p<0.05 vs control based on Student's two sided t-test).

FIG. 3 shows that treatment with F10 significantly reduces the growth of established PC3 cell xenografts. (A) Tumor growth curves for the four tumor groups in the study (left and right flank tumors from each treatment group were analyzed separately). Error bars indicate +/−SEM, n=8. (B) A typical mouse in the study. Tumors were initiated by s.c. injection of PC3 cells bilaterally. Initial tumor volumes were approximately 500 mm³ in all treatment groups. The left flank tumor in each mouse was selectively irradiated using the orthovoltage X-ray irradiator (GE Healthcare). (C) Treatment schedule for the in vivo experiment. Mice were assigned to one of two treatment groups—either F10 at 40 mg/kg dissolved in 100 μL of sterile saline or saline-only. Treatment was administered through a catheter inserted into the jugular vein. Drug was administered 3× per week for five weeks on the days indicated. A dose of 3 Gy radiation was administered to the left flank of all animals 2× per week for five weeks on the indicated days. (D) Kaplan-Meier survival curves for mice treated with F10 or with saline control. Mice were removed from the study based on tumor size in the non-irradiated flank.

FIG. 4 shows that F10 treatment does not result in weight loss greater than control treatment or cause damage to the colonic epithelium. Also, the combination of F10+radiation results in significantly smaller final tumor size relative to treatment with radiation alone. (A) Average weights for F10-treated and control mice. Error bars indicate +/−SEM, n=8. (B) Graph of the final mean tumor volumes for left-flank tumors in the study. Tumors treated with F10+irradiation were significantly smaller than tumors treated with radiation. Graph depicts the mean+/−SEM (n=8) for left flank tumors from the F10-treated and saline groups. (C) H&E section (10×) of colonic epithelium from an F10-treated mouse. (D) Colonic epithelium from a saline-treated mouse. Both mice were sacrificed as a result of tumor burden to the non-irradiated flank. There is no deterioration of the colonic epithelium in mice from treated with F10.

FIG. 5 shows that H&E sections (4×) from a left-flank (irradiated) tumor from an (A) F10-treated animal; and (B) Saline-treated animal. Irradiated tumors from the F10-treated group displayed marked hypocellularity relative to saline-treated animals. (C) H&E section from the right-flank (non-irradiated) tumor from an F10-treated animal and (D) from the right-flank of a saline-treated animal. While animals from both groups were sacrificed based on volumes in the right-flank tumors, tumors from the saline group displayed marked necrosis. Animals from the F10-treated group were sacrificed, on average, 18 days later than animals in the saline group (see FIG. 3).

### DETAILED DESCRIPTION OF THE INVENTION

The present invention relates to compounds and compositions (e.g., pharmaceutical compositions) for treating cancer and methods associated therewith. For example, the compounds, compositions and methods of the present invention can be used in the treatment of prostate cancer such as castration-resistant prostate cancer. It should be understood that if a composition or a particular compound is discussed, that the methods of using that compound and/or composition for the treatment of cancer is considered.

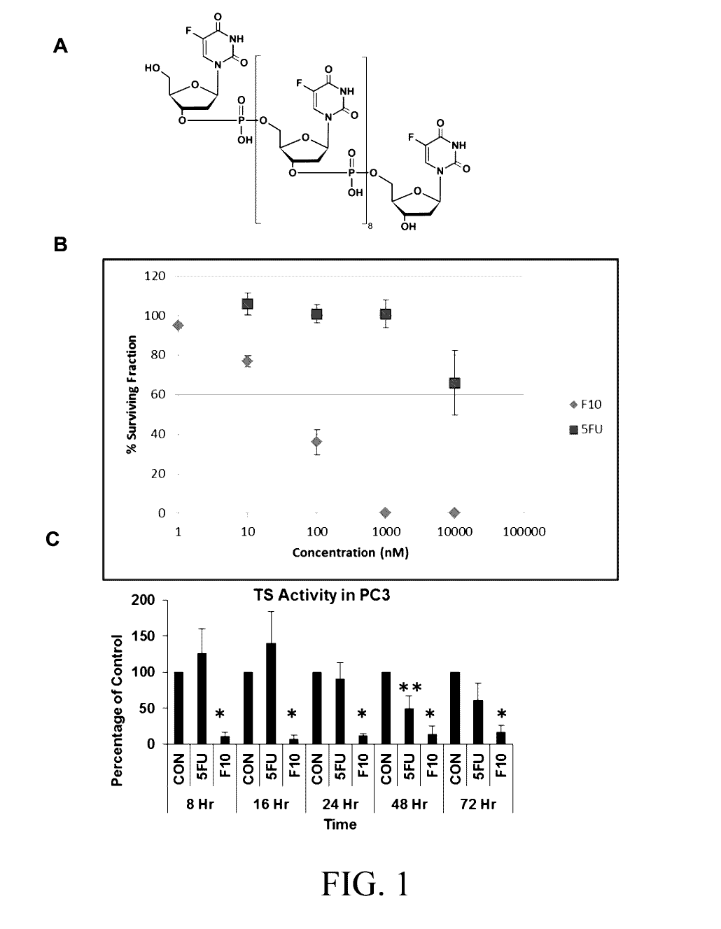

In an embodiment, the present invention relates to a composition that can be used in combination therapy for treating a plurality of cancers. For example, in one embodiment, the compound F10 (shown in FIG. 1) can be used in combination with radiation therapy to treat any of a plurality of cancers. The present invention shows that F10 leads to sensitization of the cancer cells that makes treatment with radiation much more effective relative to radiation and/or F10 used alone. For example, cancers that may be treated include brain cancer and prostate cancer.

Moreover, the survival rate of subjects that are treated by the above-enumerated combination therapy (the compound or composition in combination with radiation therapy) is greatly enhanced relative to those subjects that are treated with only the compound and/or radiation.

In an embodiment, the compounds (such as F10) of the present invention can be used in combination with radiation therapy for treating diseases of abnormal cell growth and/or dysregulated apoptosis, such as cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, ovarian cancer, cervical cancer, colon cancer, rectal cancer, cancer of the anal region, stomach cancer, gastrointestinal (gastric, colorectal, and duodenal), chronic lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular cancer (hepatic and billiary duct), primary or secondary central nervous system tumors, primary or secondary brain tumors, Hodgkin's disease, chronic or acute leukemias, chronic myeloid leukemia, lymphocytic lymphomas, lymphoblastic leukemia, follicular lymphoma, lymphoid malignancies of T-cell or B-cell origin, melanoma, multiple myeloma, oral cancer, ovarian cancer, non-small cell lung cancer, prostate cancer, small cell lung cancer, cancer of the kidney and ureter, renal cell carcinoma, carcinoma of the renal pelvis, neoplasms of the central nervous system, primary central nervous system lymphoma, non-Hodgkin's lymphoma, spinal axis tumors, brains stem glioma, pituitary adenoma, adrenocortical cancer, gall bladder cancer, cancer of the spleen, cholangiocarcinoma, fibrosarcoma, neuroblastoma, retinoblastoma, or a combination thereof.

In an embodiment, the present invention relates to a synergistic effect in combination therapy wherein the use of the compounds and/or compositions (such as F10) in combination with radiation therapy leads to an effect that is greater than the sum of the individual treatment methodologies.

In an embodiment, the present invention relates to compounds (such as F10) that are able to sensitize cancer cells (such as prostate cancer cells, or PC3 cells (a human prostate cancer cell line)) to radiation. Moreover, the compounds (such as F10) were also shown to be potent radiosensitizers of PC3 xenografts in vivo with F10 in combination with radiation resulting in significantly greater regression of PC3 xenografts than radiation alone.

It should be understood that the present invention also relates to pharmaceutical compositions that may contain pharmaceutically acceptable salts, solvates, and prodrugs thereof, and may contain diluents, excipients, carriers, or other substances necessary to increase the bioavailability or extend the lifetime of the compounds of the present invention.

Subjects that may be treated by the compounds, compositions, and methods of the present invention include, but are not limited to, horses, cows, sheep, pigs, mice, dogs, cats, primates such as chimpanzees, gorillas, rhesus monkeys, and, humans. In an embodiment, a subject is a human in need of cancer treatment.

The pharmaceutical compositions containing a compound of the invention may be in a form suitable for injection either by itself or alternatively, using liposomes, micelles, and/or nanospheres.

It is also contemplated and therefore within the scope of the invention that other anti-neoplastic agents/compounds can be used in conjunction with the compounds, compositions, methods and combination therapy of the present invention. The anti-neoplastic agents/compounds that can be used with the compounds, compositions, methods and combination therapy of the present invention include cytotoxic compounds as well as non-cytotoxic compounds.

Examples include anti-tumor agents such as HERCEPTIN™ (trastuzumab), RITUXAN™ (rituximab), ZEVALIN™ (ibritumomab tiuxetan), LYMPHOCIDE™ (epratuzumab), GLEEVEC™ and BEXXAR™ (iodine 131 tositumomab).

Other anti-neoplastic agents/compounds that can be used in conjunction with the compounds, compositions, methods and combination therapy of the present invention include anti-angiogenic compounds such as ERBITUX™ (IMC-C225), KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTIN™ or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as ABX-EGF (panitumumab), IRESSA™ (gefitinib), TARCEVA™ (erlotinib), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto).

Other anti-angiogenic compounds/agents that can be used in conjunction the compounds, compositions, methods and combination therapy of the present invention include Campath, IL-8, B-FGF, Tek antagonists, anti-TWEAK agents (e.g., specifically binding antibodies or antigen binding regions, or soluble TWEAK receptor antagonists, ADAM distintegrin domain to antagonize the binding of integrin to its ligands, specifically binding anti-eph receptor and/or anti-ephrin antibodies or antigen binding regions, and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto).

Other anti-angiogenic/anti-tumor agents that can be used in conjunction with the compounds, compositions, methods and combination therapy of the present invention include: SD-7784 (Pfizer, USA); cilengitide. (Merck KGaA, Germany, EPO 770622); pegaptanib octasodium, (Gilead Sciences, USA); Alphastatin, (BioActa, UK); M-PGA, (Celgene, USA); ilomastat, (Arriva, USA,); emaxanib, (Pfizer, USA,); vatalanib, (Novartis, Switzerland); 2-methoxyestradiol, (EntreMed, USA); TLC ELL-12, (Elan, Ireland); anecortave acetate, (Alcon, USA); alpha-D148 Mab, (Amgen, USA); CEP-7055, (Cephalon, USA); anti-Vn Mab, (Crucell, Netherlands) DAC:antiangiogenic, (ConjuChem, Canada); Angiocidin, (InKine Pharmaceutical, USA); KM-2550, (Kyowa Hakko, Japan); SU-0879, (Pfizer, USA); CGP-79787, (Novartis, Switzerland); the ARGENT technology of Ariad, USA; YIGSR-Stealth, (Johnson & Johnson, USA); fibrinogen-E fragment, (BioActa, UK); the angiogenesis inhibitors of Trigen, UK; TBC-1635, (Encysive Pharmaceuticals, USA); SC-236, (Pfizer, USA); ABT-567, (Abbott, USA); Metastatin, (EntreMed, USA); angiogenesis inhibitor, (Tripep, Sweden); maspin, (Sosei, Japan); 2-methoxyestradiol, (Oncology Sciences Corporation, USA); ER-68203-00, (WVAX, USA); Benefin, (Lane Labs, USA); Tz-93, (Tsumura, Japan); TAN-1120, (Takeda, Japan); FR-111142, (Fujisawa, Japan); platelet factor 4, (RepliGen, USA); vascular endothelial growth factor antagonist, (Borean, Denmark); bevacizumab (pINN), (Genentech, USA); XL 784, (Exelixis, USA); XL 647, (Exelixis, USA); MAb, alpha5beta3 integrin, second generation, (Applied Molecular Evolution, USA and MedImmune, USA); gene therapy, retinopathy, (Oxford BioMedica, UK); enzastaurin hydrochloride (USAN), (Lilly, USA); CEP 7055, (Cephalon, USA and Sanofi-Synthelabo, France); BC 1, (Genoa Institute of Cancer Research, Italy); angiogenesis inhibitor, (Alchemia, Australia); VEGF antagonist, (Regeneron, USA); rBPI 21 and BPI-derived antiangiogenic, (XOMA, USA); PI 88, (Progen, Australia); cilengitide (pINN), (Merck KGaA, German; Munich Technical University, Germany, Scripps Clinic and Research Foundation, USA); cetuximab (INN), (Aventis, France); AVE 8062, (Ajinomoto, Japan); AS 1404, (Cancer Research Laboratory, New Zealand); SG 292, (Telios, USA); Endostatin, (Boston Children's Hospital, USA); ATN 161, (Attenuon, USA); ANGIOSTATIN, (Boston Children's Hospital, USA); 2-methoxyestradiol, (Boston Children's Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (p1NN), (GlaxoSmithKline, UK); E 7820, (Eisai, Japan); CYC 381, (Harvard University, USA); AE 941, (Aeterna, Canada); vaccine, angiogenesis, (EntreMed, USA); urokinase plasminogen activator inhibitor, (Dendreon, USA); oglufanide (pINN), (Melmotte, USA); HIF-1alfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol, (EntreMed, USA); anginex, (Maastricht University, Netherlands, and Minnesota University, USA); ABT 510, (Abbott, USA); AAL 993, (Novartis, Switzerland); VEGI, (ProteomTech, USA); tumor necrosis factor-alpha inhibitors, (National Institute on Aging, USA); SU 11248, (Pfizer, USA and SUGEN USA); ABT 518, (Abbott, USA); YH16, (Yantai Rongchang, China); S-3APG, (Boston Children's Hospital, USA and EntreMed, USA); MAb, KDR, (ImClone Systems, USA); MAb, alpha5 beta1, (Protein Design, USA); KDR kinase inhibitor, (Celltech Group, UK, and Johnson & Johnson, USA); GFB 116, (South Florida University, USA and Yale University, USA); CS 706, (Sankyo, Japan); combretastatin A4 prodrugs, (Arizona State University, USA); chondroitinase AC, (IBEX, Canada); BAY RES 2690, (Bayer, Germany); AGM 1470, (Harvard University, USA, Takeda, Japan, and TAP, USA); AG 13925, (Agouron, USA); Tetrathiomolybdate, (University of Michigan, USA); GCS 100, (Wayne State University, USA) CV 247, (Ivy Medical, UK); CKD 732, (Chong Kun Dang, South Korea); MAb, vascular endothelium growth factor, (Xenova, UK); irsogladine (INN), (Nippon Shinyaku, Japan); RG 13577, (Aventis, France); WX 360, (Wilex, Germany); squalamine (pIN), (Genaera, USA); RPI 4610, (Sima, USA); heparanase inhibitors, (InSight, Israel); KL 3106, (Kolon, South Korea); Honokiol, (Emory University, USA); ZK CDK, (Schering AG, Germany); ZK Angio, (Schering AG, Germany); ZK 229561, (Novartis, Switzerland, and Schering AG, Germany); XMP 300, (XOMA, USA); VGA 1102, (Taisho, Japan); VEGF receptor modulators, (Pharmacopeia, USA); VE-cadherin-2 antagonists, (ImClone Systems, USA); Vasostatin, (National Institutes of Health, USA); vaccine, Flk-1, (ImClone Systems, USA); TZ 93, (Tsumura, Japan); TumStatin, (Beth Israel Hospital, USA); truncated soluble FLT 1 (vascular endothelial growth factor receptor 1), (Merck & Co, USA); Tie-2 ligands, (Regeneron, USA); and, thrombospondin 1 inhibitor, (Allegheny Health, Education and Research Foundation, USA).
Culture and Reagents. The human prostate cancer cell line PC3 was obtained from the American Type Culture Collection (Rockville, Md.). PC3 cells were cultured at 37° C. in 5% CO₂ atmosphere in RPMI-1640 medium (Gibco/Invitrogen, Carlsbad, Calif.) supplemented with 10% fetal bovine serum, 2 mM L-glutamine, and 1% penicillin-streptomycin. Cells were passaged every 3 to 5 days upon reaching 80% confluence using 0.25% trypsin/0.05% EDTA. F10 was synthesized and purified under a National Cancer Institute (NCI) contract to support the RAID project. Concentrations of F10 solutions were determined from UV absorption at 260 nm using 30 μg/OD.
TS Catalytic Activity Assays. PC3 cells were plated at a density of 1.5×10⁶ cells in 100 mm² plates. Cells were grown overnight in RPMI 1640 medium with 10% FBS. Cells were treated with 5-FU or F10 at the indicated concentrations and incubated for 0-48 h, harvested, and lysed by freeze-fracturing. Following centrifugation of cell lysates, supernatants were assayed for protein content and TS catalytic activity.
Clonogenic Assay. PC3 cells were cultured as described above, and were passaged three days prior to plating the cells in 60×15 mm Petri dishes for the clonogenic assays. Cells at 500 and 750 cells/mL were plated for F10 concentrations of 10⁻¹⁰ to 10⁻⁶ M while 250 and 500 cell/mL were plated for 5-FU concentrations from 10⁻⁸ to 10⁻⁵ M. Cells were allowed to attach for 24 h prior to treatment for 72 h. Each experiment was done in duplicate. The mean value and standard deviation were determined for each drug concentration. Following treatment, the medium was removed and replaced with fresh medium. Cells were then incubated for seven days, and stained with crystal violet. Colonies were counted manually, with a minimal colony diameter of approximately 1 mm required for counting.
Radiation Enhancement. The effects of F10 on the response of PC3 cells to radiation were also evaluated using clonogenic assay. PC3 cells were plated as described above for F10-only treatment at densities of 200 and 400 cells/mL (0 Gy), 400 and 800 cells/mL (2 Gy), 800 and 1,600 cells/mL (4 Gy), 1,600 and 3,200 cells/mL (6 Gy) and 3,200 and 6,400 cells/mL (8 Gy). Cells were incubated with F10 for 24 h and then irradiated using a 300 kV orthovoltage X-Ray irradiator (Precision X-Ray, incorporated, North Brantford, Conn.). Following irradiation, cells were further incubated to complete the entire drug exposure period of 72 h. The surviving fraction was normalized to F10-only treatment to determine to what extent F10 enhanced radiation-mediated cytotoxicity apart from drug-induced effects.
Jugular Vein Catheterization. Male NCr Nude (nu/nu) mice were purchased from The National Cancer Institute (Bethesda, Md.) and maintained in a WFSM animal facility. All treatments and procedures in mice were conducted according to guidelines approved by the Animal Care and Use Committee of Wake Forest University Health Sciences. Prior to tumor inoculation, polyethylene tubing (PE10) was used to cannulate the jugular vein of mice seven weeks of age. The portion of the catheter, which exited through the animal's skin, was 4-5 cm in length, allowing multiple procedures during the course of the study. Catheters were supplied with a heparin lock and heat-sealed following each procedure. A two component, cloth-covered button kit was used to contain and protect the portion of the catheter outside the mouse. The button was sewn to the mid-scapular region on the mouse's back with the base attached to the mouse and the heat-sealed catheter protected by a cap. For each saline or drug injection, the cap was removed, the catheter was wiped with an alcohol pad and the heat seal removed with a sterile blade. A 30-gauge needle was used to flush the line first with heparinized saline, followed by administration of saline or drug, and followed with a heparin lock and heat-sealing. All injections and all irradiation procedures were carried out with the mice anesthetized with isoflurane. Mice were placed in a Plexiglass induction chamber (25×11×12 cm) that was filled with 2% isoflurane in O₂. Pedal reflex was tested when the mice showed no eye-blink response when the chamber was tapped (˜2 min). Anesthesia was maintained through voluntary breathing of a mixture of 1.5-2% isofluorane in O₂.
Establishment of tumor xenografts and in vivo experiments. The human prostate cancer cell line PC3 was grown to 80% confluence and harvested. Cells were re-suspended in serum-free RPMI-1640 medium with penicillin and streptomycin, mixed 1:1 with Growth Factor Reduced (GFR) BD Matrigel Basement Membrane Matrix (BD Biosciences, Palo Alto, Calif.). Using a cold syringe and 27-gauge needle, 3.5×10⁶ cells were injected subcutaneously into each lateral flank of male athymic nu/nu mice 8 weeks of age. At two weeks post-inoculation, palpable tumors (˜500 mm³) were established and animals were randomized into control and treatment arms consisting of eight to nine animals, respectively, with the latter receiving 40 mg/kg body weight F10 in 100 μL of sterile saline intravenously via jugular vein catheters beginning on Day 0 and alternating with orthovoltage (X-Ray) radiation every 24 hours for five days and continuing each week for 5 weeks. An irradiation schedule of 30 Gy in 10 fractions of 3 Gy each over a 5-week period was used (2 fractions per week), delivered using a 300 kV orthovoltage X-Ray irradiator (Precision X-Ray, incorporated, North Brantford, Conn.). This irradiation schedule is equivalent to a fractionated dose regimen of approximately 36 Gy in 18 fractions of 2 Gy each or a single fraction of 12-15 Gy. Animal weights were measured each week, and perpendicular tumor diameters were measured using a Vernier scale caliper twice per week until animals were sacrificed, at which time tumors and tissues were harvested. Tumor volume was estimated using the formula: π/6×length×width×thickness. Animals were euthanized and tumors and other tissues were removed when tumor volume in a single flank exceeded 3000 mm³. Sections of the tumor were fixed in fresh 10% neutral buffered formaldehyde before embedding in paraffin for hematoxylin and eosin staining.
Statistical Methods. Survival data and tumor growth curves were compared between groups using parametric and non-parametric methods (i.e. Kaplan-Meier curves and log-rank tests) using SAS (Version 9, Cary, N.C.). Mean tumor volumes and SE values are shown in figures for comparison and to show trends. Comparisons between the four groups (drug+/−radiation; saline+/−radiation) were made using repeated measures mixed model analysis where mice were considered as random effects and day, group and the day by group interaction were included as fixed effects. The day by group interaction was examined to determine whether the four groups had different changes in tumor volumes over time. In addition to this interaction test, we compared the mean values at different time points to determine at which time longitudinally the four groups began to differ from each other. Median and ranges of tumor volumes at a given date were calculated to determine T/C ratios and these median volume scores were compared using non-parametric two-sample median tests. Survival curves were generated to compare groups and since all animals died at some time during the experiment mean survival times could be compared using two-sample t-tests (i.e. there were no censored data), as well as the median survival times (via the log-rank test).

### Results

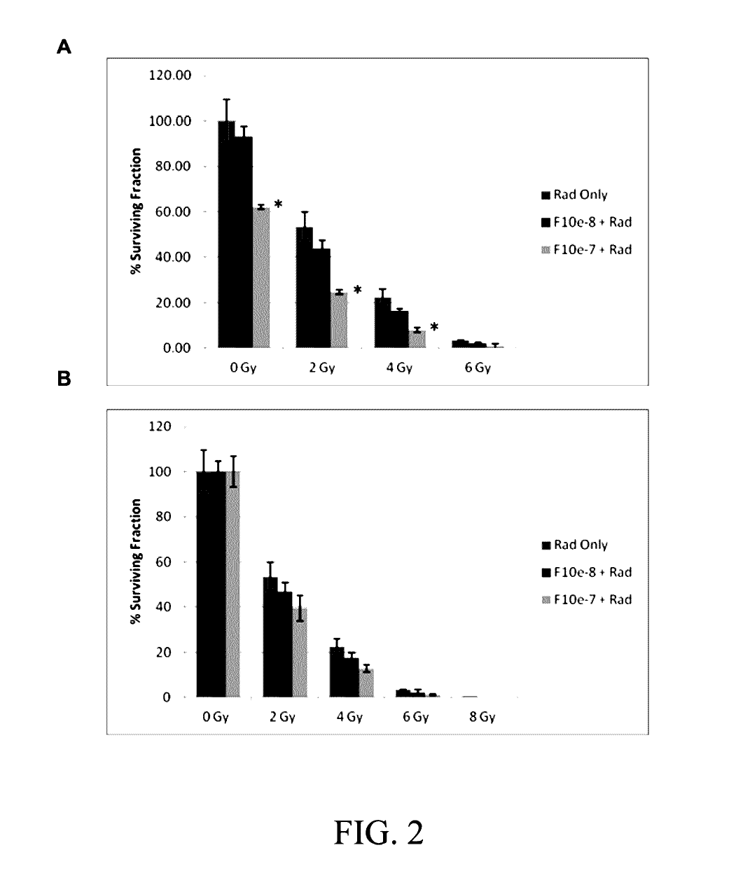

F10 Inhibits Clonogenic Survival. The NCI-60 data indicated the CRPC cell lines PC3 and DU145 were highly sensitive to F10 with GI₅₀ values in the nanomolar range. To evaluate the cytotoxicity of F10 towards PC3 cells, clonogenic assays were performed (FIG. 1). Treatment with F10 at 100 nM reduced PC3 clonogenic survival >50% while at 1 μM F10 completely inhibited PC3 cell colony formation. In contrast to the results obtained with F10, 5-FU had minimal effect on the clonogenic potential of PC3 cells. Treatment with 1 μM 5FU had no effect on PC3 cell colony formation and even 10 μM 5-FU did not decrease colony formation by 50%. These results are consistent with clinical studies demonstrating that 5-FU is unlikely to be effective for treating prostate cancer but demonstrate that F10 is substantially more potent and should be considered for prostate cancer treatment.
F10 is a Potent Inhibitor of TS. Thymidylate synthase (TS) is a principal target of fluoropyrimidine (FP) chemotherapy and inhibiting TS is considered central to the anti-tumor activity of FP drugs. TS catalytic activity was evaluated in PC3 cells following treatment with either 5-FU or F10 (FIG. 1). F10 significantly reduced TS activity relative to control within 8 h and TS activity remained significantly decreased through 72 h. In contrast 5FU treatment had minimal effect on reducing TS activity with activity levels actually increased at 8 and 16 h for 5-FU treatment relative to control and only the 48 h timepoint showing a significant decrease (˜50% control).
F10 Enhances the Effects of Radiation. Experiments were performed in order to ascertain to what extent F10 enhanced the effects of radiation at inhibiting the clonogenic survival of PC3 cells (see FIG. 2). Radiation-only was also effective at reducing colony formation of PC3 cells with 2 Gy reducing colonies to ˜50% control. For all doses of radiation evaluated, F10 co-treatment significantly decreased clonogenic potential (FIG. 2A). For example 100 nM F10 decreased colony formation at 2 Gy from 50% to 20%. When the data were normalized to separate the F10-only cytotoxic effects from the effects of radiation (FIG. 2B) a true radiosensitization effect was apparent for F10. Thus, F10 has potential to be used both for direct anti-tumor effects as well as radiosensitization for treatment of prostate cancer.

### F10 Inhibits PC3 Tumor Growth and Increases Survival

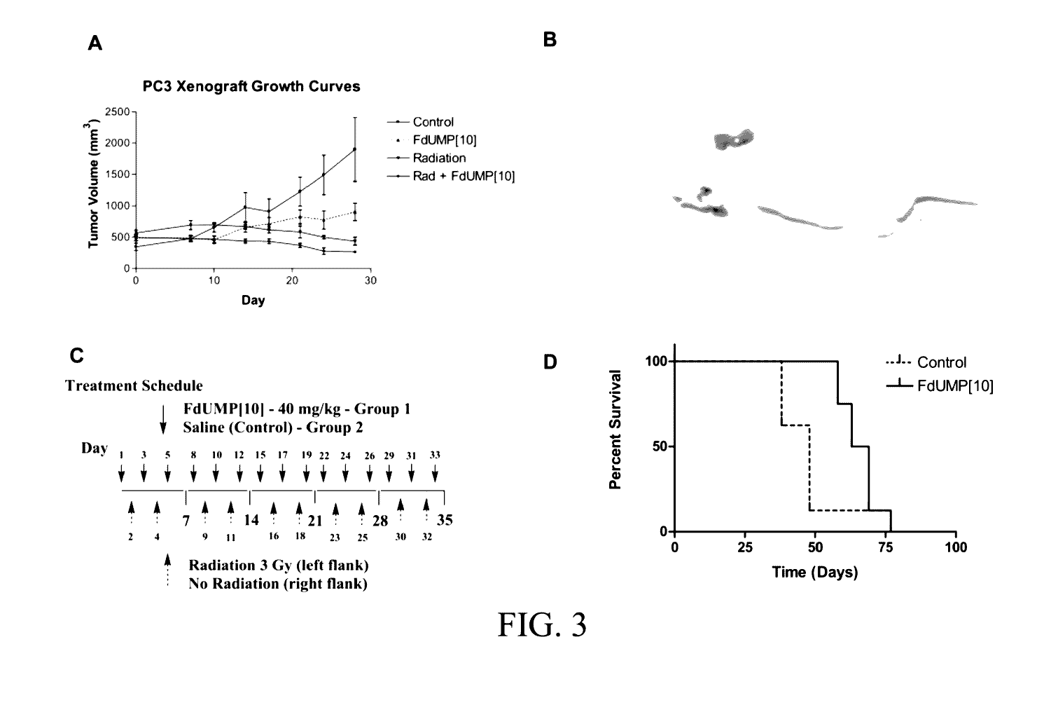

The antitumor activity of F10 was evaluated in NCR nu/nu mice in which PC3 tumor cells had been implanted 14 days previously. Mean initial tumor volumes were approximately 500 mm³. Mice treated with F10 had significantly longer survival times relative to saline-treated controls. The mean survival time for mice treated with F10 was 66 days while the mean survival time for saline-treated control animals was 48 days. Thus, treatment with F10 resulted in increased survival of 18 days (T/C days=18; p<0.001; n=16—FIG. 3). In all cases, mice were removed from the study as a result of tumor burden in the non-irradiated flank, thus survival is a direct comparison of the ability of F10 alone to reduce tumor growth. Median survival times were also significantly increased as a result of F10 treatment (p<0.002; n=16).

The repeated measures mixed model indicated that there were significant group by day interactions suggesting that the tumor volumes were changing at different rates in the four treatment groups (saline (S); F10 (F); S+radiation (R); and F10+radiation (F+R). The non-irradiated groups (F and S) began to differ starting at day 20 while the irradiated and non-irradiated (Saline) and the irradiated and non-irradiated (F10) groups began differing significantly at Day 17 (FIG. 3). The median tumor volumes were compared between groups and it was found using one-tailed comparisons that by day 24 there were significant differences between all groups. The F-irradiated group (F+R) differed from the S-non-irradiated group (S) (p=0.002; n=16), F-non-irradiated (F) versus S-non-irradiated (S) (p=0.026; n=16), and S-irradiated (R) versus S-non-irradiated (S) (p=0.0018; n=16). Although the growth trends clearly indicated continued differentiation among tumor size in the four treatment groups, animals with the largest tumors in the saline group were euthanized due to tumor burden in the non-irradiated flank and direct comparisons among treatment groups were not possible at later timepoints.

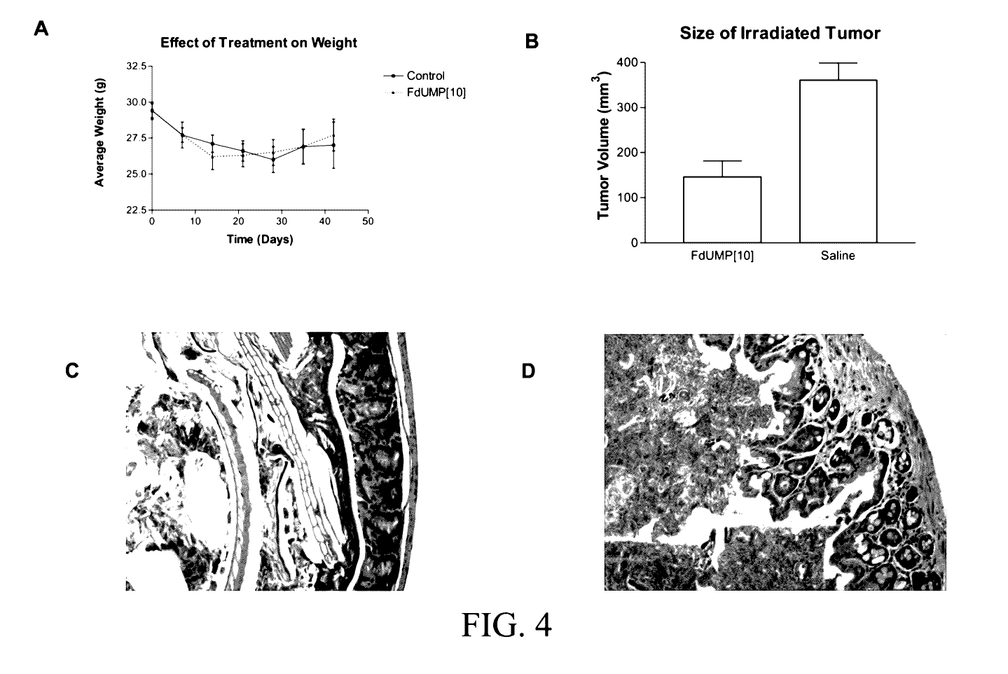

Treatment with F10 did not result in weight loss significantly greater than mice treated with vehicle-only. The weight loss in the F10-treated mice was greatest on day 14 with a mean weight loss of 11%. Saline-treated animals had a mean weight loss of 8% at day 14 and a mean weight loss of 11% on day 28 (FIG. 4). The weight loss displayed in both the drug-treated and non-drug-treated mice likely resulted from the effects of the anesthesia (isoflurane) that was administered daily prior to either drug-injection or irradiation. The F10-treated mice quickly adjusted to the treatment protocol and began re-gaining weight during the third week of treatment. In contrast, the saline-treated control group continued to lose weight as tumor burden increased in these animals. The present results confirm that extended treatment with F10 is very well-tolerated in vivo and does not result in significant weight-loss at efficacious doses.

Histological examination of tissues from animals sacrificed at the conclusion of the study revealed no toxicity to the gastrointestinal tract or to other tissues of drug-treated mice (FIG. 4). Mice-treated with F10 also displayed no signs of neutropenia as assessed by histological examination of a cross-section of the femur (data not shown). Other tissues examined included liver, lungs, and kidneys. There was no sign of drug-related toxicity in any tissues. Thus, the results indicate that F10 at a concentration of 40 mg/kg/dose significantly reduces tumor growth; furthermore, doses that are higher than those administered in the present study are likely to be well-tolerated in vivo. Since a dose-response is evident for PC3 cells in culture, it is possible that an even greater reduction in tumor burden could be achieved with higher dosage.

### The Combination of F10 and Radiation Potently Reduces Tumor Burden

The antitumor activity of F10 in combination with radiation was compared to radiation only, drug-treatment alone, and no treatment by analyzing the growth curves for the left-flank tumors of mice treated with F10 relative to the other tumor groups (FIG. 3). The growth rates for tumors treated with F10+radiation were significantly less than for tumors treated with radiation-only, drug-only, or saline control. The tumor growth delay (T/C %=21%) for the F10+radiation group was significantly reduced relative to control (p<0.005; n=16). The tumor growth delay for radiation only was also significant relative to control (T/C %=36%; p<0.05; n=16). The radiation-sensitizing properties of F10 were apparent in comparison of the F10+radiation group to radiation alone (p<0.01; n=16).

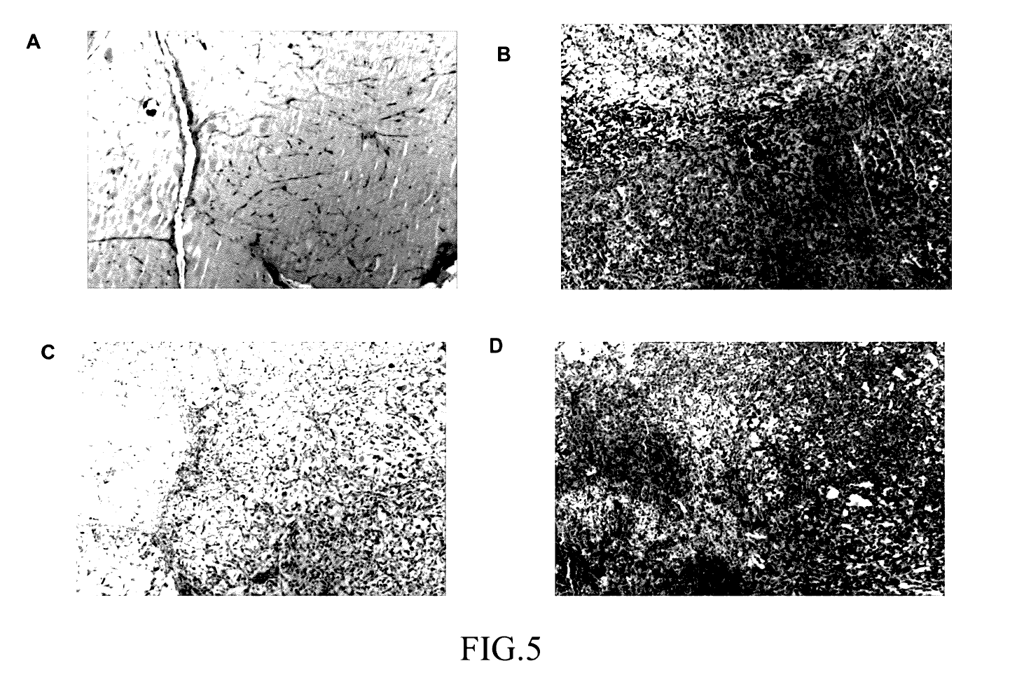

Although the study design did not permit direct comparison with respect to survival of mice treated with F10+radiation relative to radiation-only and to drug-only, the final tumor sizes for PC3 xenografts treated with F10+radiation were significantly smaller, on average, than were the final tumor sizes for PC3 xenografts treated with radiation only (FIG. 4). Measurement of final tumor size for the F10+radiation PC3 xenografts occurred an average of 18 days later than for xenografts treated with radiation-only, as euthanasia of animals was required based on tumor volume for the non-irradiated flank. Average tumor volumes did not become significantly larger for the tumors treated with F10+radiation during the final weeks of the study indicating that the combination had a long-term effect on tumor growth and animal survival. Histological examination of tumors treated with F10+radiation revealed marked hypocellularity of the excised tissue (FIG. 5). The results indicate that F10 is a potent radiosensitizer and that the combination of F10+radiation may be highly effective for the treatment of prostate cancer. Histological examination of tumors from the non-irradiated flank of saline-treated and F10-treated mice revealed marked necrosis in the saline-treated animals, but not the F10-treated mice which were euthanized, on average, 18 days later. The results are consistent with tumor re-growth following the conclusion of F10 administration on day 33 (FIG. 3).

Advanced prostate cancer remains a challenging disease with few effective chemotherapeutic options. The inventors studies show the novel FP F10 increases survival of mice with PC3 xenografts and sensitizes PC3 xenografts to radiation. These results are somewhat unexpected in light of previous studies reporting that 5-FU and capecitabine are not efficacious for treating prostate cancer and likely reflects selective targeting of the DNA-directed locus of FP activity. Thus, F10 may be effective in the clinical management of prostate cancer both as a chemotherapeutic agent and as a radiosensitizer.

The results of the present study stand in stark contrast to previous studies with 5-FU that determined the maximum tolerated dose of 5-FU to be 45 mg/kg/dose on a once daily, three times per week dosing schedule with higher doses resulting in lethality. In an embodiment of the present invention, dosing with F10 at 40 mg/kg/dose 3-times per week for 5 consecutive weeks could be continued with no adverse drug-related effects. Animals in the F10-treated group experienced no drug-induced weight loss and histological examination of the GI-tract following animal sacrifice at the end of the study indicated no drug-related damage to the GI-tract or any other tissues. These results are consistent with the GI-tract toxicity of FPs being mainly an RNA-mediated effect and indicate that selectively targeting the DNA-directed locus of FP activity with F10 results in significant antitumor activity and with elimination of the GI-tract toxicity associated with 5-FU treatment. The lack of toxicity at the current dose indicates that higher or more extensive dosing than was employed in the present study is likely to be safe. Subsequently doses as high as 300 mg/kg were administered safely. In light of the observed concentration-dependence of F10 cytotoxicity and radiosensitization, higher dosing may further reduce tumor burden in vivo.

Thus, in an embodiment, the present invention relates to doses that are close to, at, or higher than the dose that those of skill in the art thought might be the maximal dose. In a variation, the dose level may be 45 mg/kg/dose on a once daily, three times per week for multiple weeks and this dosage level may be combined with radiation therapy. Alternatively, the dose level may be 40 mg/kg/dose on a once daily, three times per week for multiple weeks (3 weeks, or 4 weeks, or 5 weeks, or 6 weeks or longer), or alternatively, the dose level may be 50 mg/kg/dose or 60 mg/kg/dose or up to 200 mg/kg/dose on a once daily, three times per week for multiple weeks. Doses as high as 300 mg/kg have been administered safely. Thus, even higher doses may be used and tolerated.

F10 exhibits remarkable activity against PC3 xenografts. Thus, in an embodiment, the present invention relates to a composition that can be administered to an individual and can be used to treat prostate cancer. In one variation, the composition comprises an oligonucleotide which in one variation comprises 5-fluoro-2′-deoxyuridine-5′-O-monophosphate. In one variation, the oligonucleotide(s) comprise(s) F10. In one variation, it is contemplated and therefore within the scope of the invention that the oligonucleotide length can be a 3-20 mer, or alternatively, a 6-18 mer. In one variation, it is contemplated that F10 is part of a larger molecule (which means that the length of the oligonucleotide is at least a 10 mer). In one variation, F10 is a part of a larger molecule with additional nucleotides and the additional nucleotides are not 5-fluoro-2′-deoxyuridine-5′-O-monophosphate. In one variation, some of the additional nucleotides may be 5-fluoro-2′-deoxyuridine-5′-O-monophosphate. In another variation, all of the additional nucleotides are 5-fluoro-2′-deoxyuridine-5′-O-monophosphate.

In one variation, F10 may be covalently linked to another molecule that is not a nucleotide. For example, antibodies or phospholipids may be used.

In an embodiment, the composition is administered to an individual at a dosage wherein F10 is present at a concentration between about 5 mg/kg and 80 mg/kg, or alternatively, at a dosage wherein F10 is present at a concentration between about 10 mg/kg and 70 mg/kg, or alternatively, at a dosage wherein F10 is present at a concentration between about 20 mg/kg and 65 mg/kg, or alternatively, at a dosage wherein F10 is present at a concentration between about 30 mg/kg and 50 mg/kg, or alternatively, at a dosage wherein F10 is present at a concentration at about 40 mg/kg. In one variation, the composition may also comprise a pharmaceutically acceptable diluent, carrier, or excipient.

“Treat” as used herein refers to any type of treatment that imparts a benefit to a subject or patient, including but not limited to reducing symptoms, eliminating symptoms, delaying the onset of symptoms, slowing the rate of progression of symptoms, etc.

Likewise, a “pharmaceutically effective” dose/amount is an amount/dosage that is sufficient to impart a benefit to a subject or patient, including but not limited to reducing symptoms, eliminating symptoms, delaying the onset of symptoms, slowing the rate of progression of symptoms, etc.

“Pharmaceutically acceptable” as used herein means that the compound or composition is suitable for administration to a subject to achieve the treatments described herein, without unduly deleterious side effects in light of the severity of the disease and necessity of the treatment.

The term “about” when used in conjunction with the dosage means a range of +/−15 mg/kg. However, it is contemplated that other error bars may be used, for example, when a dosage is administered, it is contemplated that the error bar may be +/−10 mg/kg, or alternatively, +/−5 mg/kg, or alternatively, +/−3 mg/kg, or alternatively, +/−1 mg/kg.

In one variation, the subject may be treated with a pharmaceutically acceptable amount of oligonucleotide (such as F10) by intravenous injection every other day (QOD) for 3, 4, 6 or 9 treatments. In a variation, the treatment regime may comprise administering treatment every day for between about 3-20 days. Alternatively, treatment may be administered QOD for between about 3-20 treatments (e.g., up to 40 days). In one variation, any of 4, 6 or 9 treatments may be used and then treatment stop for a plurality of months (for example, 6 months, or alternatively, 12 months) whereupon treatment may be repeated for any of 4, 6 or 9 treatments (usually, QOD).

In one variation, the pharmaceutical composition and methods using the composition may contain pharmaceutically acceptable salts, solvates, and prodrugs thereof, and may contain diluents, excipients, carriers, or other substances necessary to increase the bioavailability or extend the lifetime of the compounds/oligonucleotides of the present invention.

Subjects (individuals) that may be treated by the compounds/oligonucleotides and compositions of the present invention include, but are not limited to, horses, cows, sheep, pigs, mice, dogs, cats, primates such as chimpanzees, gorillas, rhesus monkeys, and, humans. In an embodiment, a subject/individual is a human in need of cancer treatment (e.g., treatment for prostate cancer).

The treatment of the present invention may be tailored to address the age of the individual being treated. Because ALL tends to be more prevalent in human patients that are younger than age 20, accounting for 76% of all leukemia diagnosed before that age, the treatment may be tailored to treat younger patients. ALL is especially common in children younger than 5. After a child grows into adulthood, the general risk of ALL rises again after age 50. About four out of every ten ALL diagnoses will be adults. Thus, in an embodiment, the treatment may be tailored to be administered on a subject that is 20 or younger or alternatively, and/or additionally tailored to be administered to a patient that is 50 or older.

The pharmaceutical compositions containing oligonucleotides of the invention may be in a form suitable for oral use, for example, as tablets, troches, lozenges, aqueous, or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques to form osmotic therapeutic tablets for controlled release.

Formulations for oral use may also be presented as hard gelatin capsules where the active ingredient is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil.

Aqueous suspensions may contain the active oligonucleotides in an admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycethanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and/or flavoring and/or coloring agents.

The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, sterile water for injection (SWFI), Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

In one variation, the formulations of the present invention suitable for parenteral administration may comprise sterile aqueous and non-aqueous injection solutions of the active compound(s), which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s)/oligonucleotides, or a salt thereof, in a unit dosage form in a sealed container. The compound/oligonucleotides or salts thereof is provided in the form of a lyophilizate which is capable of being reconstituted with a suitable pharmaceutically acceptable carrier to form a liquid composition suitable for injection thereof into a subject. The unit dosage form may in one variation comprise from about 10 mg to about 10 grams of the compound/oligonucleotide or salt thereof. When the compound/oligonucleotide(s) or salt thereof is substantially water-insoluble, a sufficient amount of emulsifying agent which is physiologically acceptable may be employed in sufficient quantity to emulsify the compound or salt in an aqueous carrier. One such useful emulsifying agent that may be used is phosphatidyl choline.

Thus, in another embodiment, the present invention provides a pharmaceutical formulation solution comprising oligonucleotides (such as F10) or a salt thereof.

A solution of the invention may be provided in a sealed container, especially one made of glass, either in a unit dosage form or in a multiple dosage form.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

As noted above, the present invention provides pharmaceutical formulations/compositions comprising the active compounds/oligonucleotides (including the pharmaceutically acceptable salts thereof), in pharmaceutically acceptable carriers for oral, rectal, topical, buccal, parenteral, intramuscular, intradermal, intravenous, and/or transdermal administration.

Any pharmaceutically acceptable salt of oligonucleotides (such as F10) may be used for preparing a solution of the invention. Examples of suitable salts may be, for instance, the salts with mineral inorganic acids such as hydrochloric, hydrobromic, sulfuric, phosphoric, nitric and the like, and the salts with certain organic acids such as acetic, succinic, tartaric, ascorbic, citric, glutamic, benzoic, methanesulfonic, ethanesulfonic and the like. In an embodiment, the oligonucleotides (such as F10) is a hydrochloric acid salt including a mono, di, or trihydrochloride.

Any solvent which is pharmaceutically acceptable and which is able to dissolve the oligonucleotides (such as F10) or a pharmaceutically acceptable salt thereof may be used. The solution of the invention may also contain one or more additional components such as a co-solubilizing agent (which may be the same as a solvent), a tonicity adjustment agent, a stabilizing agent, a preservative, or mixtures thereof. Examples of solvents, co-solubilizing agents, tonicity adjustment agents, stabilizing agents and preservatives which may suitable for a solution formulation are described below.

Suitable solvents and co-solubilizing agents may include, but are not limited to, water; sterile water for injection (SWFI); physiological saline; alcohols, e.g. ethanol, benzyl alcohol and the like; glycols and polyalcohols, e.g. propyleneglycol, glycerin and the like; esters of polyalcohols, e.g. diacetine, triacetine and the like; polyglycols and polyethers, e.g. polyethyleneglycol 400, propyleneglycol methylethers and the like; dioxolanes, e.g. isopropylidenglycerin and the like; dimethylisosorbide; pyrrolidone derivatives, e.g. 2-pyrrolidone, N-methyl-2-pyrrolidone, polyvinylpyrrolidone (co-solubilizing agent only) and the like; polyoxyethylenated fatty alcohols; esters of polyoxyethylenated fatty acids; polysorbates, e.g., Tween™, polyoxyethylene derivatives of polypropyleneglycols, e.g., Pluronics™.

Suitable tonicity adjustment agents may include, but are not limited to, pharmaceutically acceptable inorganic chlorides, e.g. sodium chloride; dextrose; lactose; mannitol; sorbitol and the like.

Preservatives suitable for physiological administration may be, for instance, esters of parahydroxybenzoic acid (e.g., methyl, ethyl, propyl and butyl esters, or mixtures of them), chlorocresol and the like.

Suitable stabilizing agents include, but are not limited to, monosaccharides (e.g., galactose, fructose, and fucose), disaccharides (e.g., lactose), polysaccharides (e.g., dextran), cyclic oligosaccharides (e.g., alpha-, beta-, gamma-cyclodextrin), aliphatic polyols (e.g., mannitol, sorbitol, and thioglycerol), cyclic polyols (e.g. inositol) and organic solvents (e.g., ethyl alcohol and glycerol).

The above mentioned solvents and co-solubilizing agents, tonicity adjustment agents, stabilizing agents and preservatives can be used alone or as a mixture of two or more of them in a solution formulation.

In an embodiment, a pharmaceutical solution formulation may comprise an oligonucleotide (such as F10) or a pharmaceutically acceptable salt thereof, and an agent selected from the group consisting of sodium chloride solution (i.e., physiological saline), dextrose, mannitol, or sorbitol, wherein the agent is in an amount of less than or equal to 5%. The pH of such a formulation may also be adjusted to improve the storage stability using a pharmaceutically acceptable acid or base.

In the solutions of the invention the concentration of F10 or a pharmaceutically acceptable salt thereof may be less than 1000 mg/mL, or less than 500 mg/mL, or less than 400 mg/mL, or less than 300 mg/mL and greater than 0.01 mg/mL. In an embodiment, the concentration that is used is the ideal concentration to be sufficiently cytotoxic to the cancer cells yet limit the toxicity on other cells.

Suitable packaging for the pharmaceutical solution formulations may be all approved containers intended for parenteral use, such as plastic and glass containers, ready-to-use syringes and the like. In an embodiment, the container is a sealed glass container, e.g. a vial or an ampoule. A hermetically sealed glass vial is particularly preferred.

According to an embodiment of the present invention, there is provided, in a sealed glass container, a sterile, injectable solution comprising F10 or a pharmaceutically acceptable salt thereof in a physiologically acceptable solvent, and which has a pH of from 2.5 to 3.5. For solution formulations, various compounds/oligonucleotides of the present invention may be more soluble or stable for longer periods in solutions at a pH lower than 6. Further, acid salts of the compounds/oligonucleotides of the present invention may be more soluble in aqueous solutions than their free base counter parts, but when the acid salts are added to aqueous solutions the pH of the solution may be too low to be suitable for administration. Thus, solution formulations having a pH above pH 4.5 may be combined prior to administration with a diluent solution of pH greater than 7 such that the pH of the combination formulation administered is pH 4.5 or higher. In one embodiment, the diluent solution comprises a pharmaceutically acceptable base such as sodium hydroxide. In another embodiment, the diluent solution is at pH of between 10 and 12. In another embodiment, the pH of the combined formulation administered is greater than 5.0. In another embodiment, the pH of the combined formulation administered is between pH 5.0 and 7.0.

The invention also provides a process for producing a sterile solution with a pH of from 2.5 to 3.5 which process comprises dissolving F10 or a pharmaceutically acceptable salt thereof in a pharmaceutically acceptable solvent. Where a pharmaceutically acceptable acid salt of F10 is used the pH of the solution may be adjusted using a pharmaceutically acceptable base or basic solution adding a physiologically acceptable acid or buffer to adjust the pH within a desired range. The method may further comprise passing the resulting solution through a sterilizing filter.

One or more additional components such as co-solubilizing agents, tonicity adjustment agents, stabilizing agents and preservatives, for instance of the kind previously specified, may be added to the solution prior to passing the solution through the sterilizing filter.

Combination treatments that can be used in conjunction with the oligonucleotides/compounds of the present invention (for example, to treat ALL) may include chemotherapy, chemotherapy with stem cell transplant, radiation therapy and/or immunotherapy.

In a further variation, the present invention contemplates combination therapies in which the oligonucleotides (such as F10) of the present invention can be used in conjunction with other cisplatin compounds. The efficacy of this combination therapy is likely to be enhanced because of the different mechanisms and modes of action that first generation cisplatin compounds exhibit relative to the compounds/oligonucleotides of the present invention. It is also contemplated and therefore within the scope of the invention that other anti-neoplastic agents/compounds can be used in conjunction with the compounds/oligonucleotides of the present invention. The anti-neoplastic agents/compounds that can be used with the compounds/oligonucleotides of the present invention include cytotoxic compounds as well as non-cytotoxic compounds.

Examples include anti-tumor agents such as HERCEPTIN™ (trastuzumab), RITUXAN™ (rituximab), ZEVALIN™ (ibritumomab tiuxetan), LYMPHOCIDE™ (epratuzumab), GLEEVEC™ and BEXXAR™(iodine 131 tositumomab).

Other anti-neoplastic agents/compounds that can be used in conjunction with the compounds/oligonucleotides of the present invention include anti-angiogenic compounds such as ERBITUX™ (IMC-C225), KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTIN™ or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as ABX-EGF (panitumumab), IRESSA™ (gefitinib), TARCEVA™ (erlotinib), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto).

Other anti-angiogenic compounds/agents that can be used in conjunction with the oligonucleotides (such as F10) of the present invention include Campath, IL-8, B-FGF, Tek antagonists, anti-TWEAK agents (e.g., specifically binding antibodies or antigen binding regions, or soluble TWEAK receptor antagonists, ADAM distintegrin domain to antagonize the binding of integrin to its ligands, specifically binding anti-eph receptor and/or anti-ephrin antibodies or antigen binding regions, and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto).

Other anti-angiogenic/anti-tumor agents that can be used in conjunction with the oligonucleotides (such as F10) of the present invention include: SD-7784 (Pfizer, USA); cilengitide. (Merck KGaA, Germany, EPO 770622); pegaptanib octasodium, (Gilead Sciences, USA); Alphastatin, (BioActa, UK); M-PGA, (Celgene, USA); ilomastat, (Arriva, USA,); emaxanib, (Pfizer, USA,); vatalanib, (Novartis, Switzerland); 2-methoxyestradiol, (EntreMed, USA); TLC ELL-12, (Elan, Ireland); anecortave acetate, (Alcon, USA); alpha-D148 Mab, (Amgen, USA); CEP-7055, (Cephalon, USA); anti-Vn Mab, (Crucell, Netherlands) DAC:antiangiogenic, (ConjuChem, Canada); Angiocidin, (InKine Pharmaceutical, USA); KM-2550, (Kyowa Hakko, Japan); SU-0879, (Pfizer, USA); CGP-79787, (Novartis, Switzerland); the ARGENT technology of Ariad, USA; YIGSR-Stealth, (Johnson & Johnson, USA); fibrinogen-E fragment, (BioActa, UK); the angiogenesis inhibitors of Trigen, UK; TBC-1635, (Encysive Pharmaceuticals, USA); SC-236, (Pfizer, USA); ABT-567, (Abbott, USA); Metastatin, (EntreMed, USA); angiogenesis inhibitor, (Tripep, Sweden); maspin, (Sosei, Japan); 2-methoxyestradiol, (Oncology Sciences Corporation, USA); ER-68203-00, (WVAX, USA); Benefin, (Lane Labs, USA); Tz-93, (Tsumura, Japan); TAN-1120, (Takeda, Japan); FR-111142, (Fujisawa, Japan); platelet factor 4, (RepliGen, USA); vascular endothelial growth factor antagonist, (Borean, Denmark); bevacizumab (pINN), (Genentech, USA); XL 784, (Exelixis, USA); XL 647, (Exelixis, USA); MAb, alpha5beta3 integrin, second generation, (Applied Molecular Evolution, USA and MedImmune, USA); gene therapy, retinopathy, (Oxford BioMedica, UK); enzastaurin hydrochloride (USAN), (Lilly, USA); CEP 7055, (Cephalon, USA and Sanofi-Synthelabo, France); BC 1, (Genoa Institute of Cancer Research, Italy); angiogenesis inhibitor, (Alchemia, Australia); VEGF antagonist, (Regeneron, USA); rBPI 21 and BPI-derived antiangiogenic, (XOMA, USA); PI 88, (Progen, Australia); cilengitide (pINN), (Merck KGaA, German; Munich Technical University, Germany, Scripps Clinic and Research Foundation, USA); cetuximab (INN), (Aventis, France); AVE 8062, (Ajinomoto, Japan); AS 1404, (Cancer Research Laboratory, New Zealand); SG 292, (Telios, USA); Endostatin, (Boston Children's Hospital, USA); ATN 161, (Attenuon, USA); ANGIOSTATIN, (Boston Children's Hospital, USA); 2-methoxyestradiol, (Boston Children's Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (p1NN), (GlaxoSmithKline, UK); E 7820, (Eisai, Japan); CYC 381, (Harvard University, USA); AE 941, (Aeterna, Canada); vaccine, angiogenesis, (EntreMed, USA); urokinase plasminogen activator inhibitor, (Dendreon, USA); oglufanide (pINN), (Melmotte, USA); HIF-1alfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol, (EntreMed, USA); anginex, (Maastricht University, Netherlands, and Minnesota University, USA); ABT 510, (Abbott, USA); AAL 993, (Novartis, Switzerland); VEGI, (ProteomTech, USA); tumor necrosis factor-alpha inhibitors, (National Institute on Aging, USA); SU 11248, (Pfizer, USA and SUGEN USA); ABT 518, (Abbott, USA); YH16, (Yantai Rongchang, China); S-3APG, (Boston Children's Hospital, USA and EntreMed, USA); MAb, KDR, (ImClone Systems, USA); MAb, alpha5 beta1, (Protein Design, USA); KDR kinase inhibitor, (Celltech Group, UK, and Johnson & Johnson, USA); GFB 116, (South Florida University, USA and Yale University, USA); CS 706, (Sankyo, Japan); combretastatin A4 prodrugs, (Arizona State University, USA); chondroitinase AC, (IBEX, Canada); BAY RES 2690, (Bayer, Germany); AGM 1470, (Harvard University, USA, Takeda, Japan, and TAP, USA); AG 13925, (Agouron, USA); Tetrathiomolybdate, (University of Michigan, USA); GCS 100, (Wayne State University, USA) CV 247, (Ivy Medical, UK); CKD 732, (Chong Kun Dang, South Korea); MAb, vascular endothelium growth factor, (Xenova, UK); irsogladine (INN), (Nippon Shinyaku, Japan); RG 13577, (Aventis, France); WX 360, (Wilex, Germany); squalamine (pIN), (Genaera, USA); RPI 4610, (Sima, USA); heparanase inhibitors, (InSight, Israel); KL 3106, (Kolon, South Korea); Honokiol, (Emory University, USA); ZK CDK, (Schering AG, Germany); ZK Angio, (Schering AG, Germany); ZK 229561, (Novartis, Switzerland, and Schering AG, Germany); XMP 300, (XOMA, USA); VGA 1102, (Taisho, Japan); VEGF receptor modulators, (Pharmacopeia, USA); VE-cadherin-2 antagonists, (ImClone Systems, USA); Vasostatin, (National Institutes of Health, USA); vaccine, Flk-1, (ImClone Systems, USA); TZ 93, (Tsumura, Japan); TumStatin, (Beth Israel Hospital, USA); truncated soluble FLT 1 (vascular endothelial growth factor receptor 1), (Merck & Co, USA); Tie-2 ligands, (Regeneron, USA); and, thrombospondin 1 inhibitor, (Allegheny Health, Education and Research Foundation, USA).

In an embodiment, the nucleotides of the present invention can be combined with radiation therapy that uses high-energy radiation to shrink tumors and kill cancer cells (1). In an embodiment, one or more of the following members of the electromagnetic spectrum can be used: X-rays, and/or gamma rays. This radiation therapy can be combined with or used separately from charged particles that provide different types of radiation used for cancer treatment.

The radiation may be delivered by a machine outside the body (external-beam radiation therapy), or it may come from radioactive material placed in the body near cancer cells (internal radiation therapy, also called brachytherapy) or alternatively, they can be used in combination.

The present invention also contemplates using systemic radiation therapy, which uses radioactive substances, such as radioactive iodine, that travel in the blood to kill cancer cells alone or with the above radiation therapies.

In one embodiment, the radiation therapy may be sometimes given with curative intent (that is, with the hope that the treatment will cure a cancer, either by eliminating a tumor, preventing cancer recurrence, or both). In one variation, the radiation therapy may be used alone or in combination with surgery, chemotherapy, or both.

In an embodiment, the radiation therapy may also be given with palliative intent to relieve symptoms and reduce the suffering caused by cancer. Examples of palliative radiation therapy are therapies to shrink tumors formed from cancer cells that have spread to the effected part of the body from another part of the body (metastases), radiation given to shrink a tumor that is pressing on an organ or a bone, which can cause pain, or radiation given to shrink a tumor near the prostate, which can interfere with a patient's ability to urinate.

The radiosensitization properties of FPs and Top1 poisons have been documented herein. The extent of radiosensitization observed with F10 in the present study compares favorably with Gossypol and curcumin—two natural products that are being evaluated as radiosensitizers for the treatment of prostate cancer and other malignancies. F10 displays strong anticancer activity as a single agent, is radiosensitizing, and is very well tolerated in vivo. While chemically and mechanistically distinct from conventional FPs, F10 has similarities to these drugs that have been used successfully in the clinic for decades.
The following references are incorporated by reference in their entireties.
1. ACS Facts and Figures, 2013.
2. Hadaschik, B. A., R. D. Sowery, and M. E. Gleave, Novel targets and approaches in advanced prostate cancer. Curr Opin Urol, 2007. 17(3): p. 182-7.
3. Tannock, I. F., R. de Wit, W. R. Berry, J. Horti, A. Pluzanska, K. N. Chi, S. Oudard, C. Theodore, N. D. James, I. Turesson, M. A. Rosenthal, and M. A. Eisenberger, Docetaxel plus prednisone or mitoxantrone plus prednisone for advanced prostate cancer. N Engl J Med, 2004. 351(15): p. 1502-12.
4. Petrylak, D. P., C. M. Tangen, M. H. Hussain, P. N. Lara, Jr., J. A. Jones, M. E. Taplin, P. A. Burch, D. Berry, C. Moinpour, M. Kohli, M. C. Benson, E. J. Small, D. Raghavan, and E. D. Crawford, Docetaxel and estramustine compared with mitoxantrone and prednisone for advanced refractory prostate cancer. N Engl J Med, 2004. 351(15): p. 1513-20.
5. Kibel, A. S., E. Rosenbaum, M. W. Kattan, J. Picus, R. Dreicer, E. A. Klein, G. S. Chatta, J. B. Nelson, R. S. DiPaola, B. J. Roth, M. S. Cookson, G. Wilding, D. F. Jarrard, T. M. Beer, C. W. Ryan, D. P. Petrylak, M. C. Benson, A. W. Partin, E. Garrett-Mayer, and M. A. Eisenberger, Adjuvant weekly docetaxel for patients with high risk prostate cancer after radical prostatectomy: a multi-institutional pilot study. J Urol, 2007. 177(5): p. 1777-81.
6. Pardee, T. S., Gomes, E., Jennings-Gee, J., Caudell, D., Gmeiner, W. H., Unique dual targeting of thymidylate synthase and topoisomerase 1 by FdUMP[10] results in high efficacy against AML and low toxicity. Blood, 2012. 119: p. 3561-3570.
7. Jennings-Gee, J., Pardee, T S, Gmeiner, W H, Replication-dependent irreversible topoisomerase 1 poisoning is responsible for FdUMP[10] anti-leukemic activity. Exp Hematol, 2013. PMID: 23085462.
8. Gmeiner, W. H., C. Lema-Tome, D. Gibo, J. Jennings-Gee, C. Milligan, and W. Debinski, Selective anti-tumor activity of the novel fluoropyrimidine polymer F10 towards G48a orthotopic GBM tumors. J Neurooncol, 2014. 116(3): p. 447-54.
9. Gmeiner, W. H., W. C. Reinhold, and Y. Pommier, Genome-wide mRNA and microRNA profiling of the NCI 60 cell-line screen and comparison of FdUMP[10] with fluorouracil, floxuridine, and topoisomerase 1 poisons. Mol Cancer Ther, 2010. 9(12): p. 3105-14.
10. Liao, Z. Y., O. Sordet, H. L. Zhang, G. Kohlhagen, S. Antony, W. H. Gmeiner, and Y. Pommier, A novel polypyrimidine antitumor agent FdUMP[10] induces thymineless death with topoisomerase I-DNA complexes. Cancer Res, 2005. 65(11): p. 4844-51.
11. Pommier, Y., E. Leo, H. Zhang, and C. Marchand, DNA topoisomerases and their poisoning by anticancer and antibacterial drugs. Chem Biol, 2010. 17(5): p. 421-33.
12. Berlin, J. D., K. J. Propert, D. Trump, G. Wilding, G. Hudes, J. Glick, P. Burch, A. Keller, and P. Loehrer, 5-Fluorouracil and leucovorin therapy in patients with hormone refractory prostate cancer: an Eastern Cooperative Oncology Group phase II study (E1889). Am J Clin Oncol, 1998. 21(2): p. 171-6.
13. Huan, S. D., S. E. Aitken, and D. J. Stewart, A phase II study of 5-fluorouracil and high dose folinic acid in cisplatin-refractory metastatic bladder cancer. Ann Oncol, 1995. 6(8): p. 836-7.
14. Morant, R., J. Bernhard, D. Dietrich, S. Gillessen, M. Bonomo, M. Borner, J. Bauer, T. Cerny, C. Rochlitz, M. Wernli, A. Gschwend, S. Hanselmann, F. Hering, and H. P. Schmid, Capecitabine in hormone-resistant metastatic prostatic carcinoma—a phase II trial. Br J Cancer, 2004. 90(7): p. 1312-7.
15. Lawrence, T. S., M. A. Davis, H. Y. Tang, and J. Maybaum, Fluorodeoxyuridine-mediated cytotoxicity and radiosensitization require S phase progression. Int J Radiat Biol, 1996. 70(3): p. 273-80.
16. Chen, A. Y., R. Chou, S. J. Shih, D. Lau, and D. Gandara, Enhancement of radiotherapy with DNA topoisomerase I-targeted drugs. Crit Rev Oncol Hematol, 2004. 50(2): p. 111-9.
17. Sailer, S. L., Radiation therapy for prostate cancer: external beam, brachytherapy, and salvage. N C Med J, 2006. 67(2): p. 149-53.
18. Xu, L., D. Yang, S. Wang, W. Tang, M. Liu, M. Davis, J. Chen, J. M. Rae, T. Lawrence, and M. E. Lippman, (−)-Gossypol enhances response to radiation therapy and results in tumor regression of human prostate cancer. Mol Cancer Ther, 2005. 4(2): p. 197-205.
19. Pritchard, D. M., A. J. Watson, C. S. Potten, A. L. Jackman, and J. A. Hickman, Inhibition by uridine but not thymidine of p53-dependent intestinal apoptosis initiated by 5-fluorouracil: evidence for the involvement of RNA perturbation. Proc Natl Acad Sci U S A, 1997. 94(5): p. 1795-9.
20. Chendil, D., R. S. Ranga, D. Meigooni, S. Sathishkumar, and M. M. Ahmed, Curcumin confers radiosensitizing effect in prostate cancer cell line PC-3. Oncogene, 2004. 23(8): p. 1599-607.

It should be understood that the present invention is not to be limited by the above description. It is contemplated and therefore within the scope of the present invention that any feature that is described above can be combined with any other feature that is described above. Moreover, it should be understood that the present invention contemplates minor modifications that can be made to the compounds, compositions and methods of the present invention. When ranges are discussed, any number that may not be explicitly disclosed but fits within the range is contemplated as an endpoint for the range. For example, if a range of 3-20 is given, every real integer that fits within that range is contemplated as an endpoint that can be used to establish a subset range (e.g., 4, 5, 6, . . . etc. . . . 19). The scope of protection to be afforded is to be determined by the claims which follow and the breadth of interpretation which the law allows.

We claim:

1. A method of treating prostate cancer in an individual in need thereof, wherein the method comprises administering to said individual a pharmaceutically effective amount of a composition comprising F10.

2. The method of claim 1, wherein the prostate cancer is acinar adenocarcinoma.

3. The method of claim 1, further comprising threating the individual with radiation therapy.

4. The method of claim 3, wherein the prostate cancer is one or more members selected from the group consisting of acinar adenocarcinoma, ductal adenocarcinoma, transitional cell (or urothelial) cancer, squamous cell cancer, carcinoid, small cell cancer, sarcomas, and sarcomatoid cancer.

5. The method of claim 3, wherein the composition that comprises F10 contains additional nucleotides covalently linked to F10.

6. The method of claim 5, wherein the individual is treated with F10 at a dosage between about 30 mg/kg and 300 mg/kg.

7. The method of claim 6, wherein the individual is treated with F10 at a dosage of about 40 mg/kg.

8. The method of claim 3, wherein the pharmaceutically effective amount of the composition is about 45 mg/kg/dose on a once daily, three times per week.

9. The method of claim 3, wherein the pharmaceutically effective amount of the composition is administered as a radiosensitizer and is combined with another anticancer drug.

10. The method of claim 3, wherein the pharmaceutically effective amount of the composition is administered parenterally.

11. The method of claim 7, wherein the individual is treated one or more times by a treatment set that comprises 3 treatments every other day (QOD).

12. The method of claim 11, wherein the treatment set comprises between 4 and 20 treatments QOD.

13. The method of claim 12, wherein the treatment set comprises 9 treatments QOD.

14. The method of claim 12, wherein the individual is treated by a plurality of treatment sets.

15. The method of claim 1, wherein the composition further comprises one or more of a pharmaceutically acceptable diluent, carrier, or excipient.

16. A pharmaceutical composition for treating acute prostate caner in an individual in need thereof comprising F10, and optionally one or more of a pharmaceutically acceptable diluent, carrier, or excipient.

17. The pharmaceutical composition of claim 16, wherein the composition is administered at a dosage that is between about 30 and 300 mg/kg.

18. The pharmaceutical composition of claim 17, wherein the composition is administered in combination with another anticancer drug.

19. The pharmaceutical composition of claim 18, wherein the composition is administered parenterally.

20. The pharmaceutical composition of claim 16, wherein the composition further comprises co-solubilizing agents, tonicity adjustment agents, stabilizing agents or preservatives.
